(19) United States
(12) Patent Application Publication
(10) Pub. No.: US 2006/0269972 A1
(43) Pub. Date: Nov. 30, 2006

(54) Method of diagnosing colorectal adenomas and cancer using infrared spectroscopy

(76) Inventors: Ian Smith, Winnipeg (CA); Ray L. Somorjai, Headingley (CA); Jon C. Meltzer, Winnipeg (CA); Brion Dolonko, Winnipeg (CA); Alexandre Nikouline, Winnipeg (CA)

Correspondence Address: GEORGE A. SEABY;SEABY & ASSOCIATES, 250 CITY CENTRE AVNUE, OTTAWA, ON, K1R6K7, CA

(21) Appl. No.: 10/568,419

(22) PCT Filed: Aug. 5, 2004
(86) PCT No.: PCT/CA04/01462

§ 371 Date: Feb. 14, 2006

Related U.S. Application Data
(60) Provisional application No. 60/494,781, filed on Aug. 14, 2003.

(51) Int. Cl. G01N 33/574; G06F 19/00; G01N 33/00

(52) U.S. Cl. 435/7.23; 436/86; 702/19

(57) ABSTRACT

Infrared spectroscopy of human stool can be used as a non-invasive method of detecting the presence of colorectal cancer and/or clinically significant adenomas. The spectrum of a patient's stool is compared with that of stool from non-cancerous subjects, observed differences in spectra being indicative of cancer and/or clinically significant adenomas. In a preferred method, the stool sample is mixed with a buffer, the resulting suspension is centrifuged and the supernatant is subjected to infrared spectroscopy. The spectra are then classified using a three-stage classification strategy.

This invention relates to a method of detecting colorectal adenomas and cancer, and in particular to a method of detecting such adenomas and cancer using near infrared spectroscopy.

Colorectal cancer is one of the most common cancers in the U.S.A. and 105,000 people were expected to develop this disease in 2003; it was also projected that 57,000 would die of this in the U.S.A in 2003. The lifetime risk that an individual in North America will develop colorectal cancer is believed to be about 5-6%. Symptoms associated with colorectal cancer, including blood in the stool, anemia, abdominal pain and alteration of bowel habits often become apparent only when the disease has advanced significantly. It is well known that prognosis for a patient depends largely on the stage of the disease at the time of diagnosis. In fact, whereas the five-year survival for a patient whose colorectal cancer is detected at an early stage is 92%, survival decreases to about 60% in patients with regional spread, and to about 6% in those with distant metastases. Accordingly, it is important to detect the precursor adenomas and cancer as early as possible to increase the chances of successful therapeutic intervention.

A screening technique preferably provides high sensitivity and specificity, low cost, safety and simplicity. Currently, digital rectal examination (DRE), fecal occult blood test (FOBT), barium enema and direct colon visualization (sigmoidoscopy and colonoscopy) screening techniques are employed.

DRE involves examining the rectum using a finger. This method detects cancers that can be palpated and are within reach of the finger. A negative DRE provides little reassurance that a patient is free of cancer, because fewer than 10% of colorectal cancers can be palpated by the examining finger.

FOBT detects hidden blood in the stool by chemical means. Although the least expensive and the simplest, the FOBT method has low sensitivity, moderate specificity and is usually not good for early detection. According to available data, a major drawback of this technique is that more than half of the cancers discovered by this method followed by x-ray or endoscopy are usually beyond the limit of early staging. A false positive rate of 10-12% is expected when the patients tested are on an unrestricted diet. Estimates of the positive predictive value range from 2.2 to 50%. The guaiac tests have a very low sensitivity, generally around 50%. The use of FOBT is based on the assumption that colorectal cancers are associated with bleeding. However, it appears that some colorectal cancers bleed intermittently and others not at all.

A barium enema involves an x-ray of the bowel using a contrast agent. The enema can be a single or double contrast. The main radiologic signs of malignancy include muscosal disruption, abrupt cut-off and shouldering and localized lesions with sharp demarcations from uninvolved areas. The estimated sensitivity of double contrast barium enema for cancer and large polyps is only about 65-75% and even lower for small adenomas. Despite its better diagnostic yield, double contrast barium enema has a false-negative rate of 2-18%. Moreover, the method involves exposure to radiation, the repeated use of which may not be safe. Perforation from barium enema is extremely uncommon, but when it happens it is can be fatal or lead to serious long term problems as a result of barium spillage into the abdominal cavity.

A variety of instruments (collectively called endoscopes) are generally used for examining the bowel. Endoscopes can be rigid or flexible with varying lengths. Flexible sigmoidoscopes are 60 cm long. A colonoscope is a 130-160 cm flexible viewing instrument for examining the entire colon. Biopsies are taken from suspicious looking areas while viewing the colon through the endoscope. The flexible sigmoidoscopy examination is limited to the left side of the colon and rectum. At least ⅓ of neoplastic tumors are believed to occur in areas proximal to the splenic flexure that are inaccessible by sigmoidoscopy. Colonoscopy has a high sensitivity, and remains the gold standard for visualization of the colon and the detection of neoplastic abnormalities. However, it is invasive, quite expensive, and exposes the subject to risks of bowel perforation.

There are a number of currently available methods for detecting cancer in its early stages. Biophysical methods such as conventional X-rays, nuclear medicine, rectilinear scanners, ultrasound, CAT and MRI all play an important role in early detection and treatment of cancer. Clinical laboratory testing for tumor markers can also be used as an aid in early cancer detection. Tumor marker tests, which aid in diagnosis, staging, disease progression, monitoring response to therapy and detection of recurrent disease, measure either tumor-associated antigens or other substances present in cancer patients. Unfortunately, most tumor marker tests do not possess sufficient specificity to be used as screening tools in a cost-effective manner. Even highly specific tests often suffer from poor predictive value, because the prevalence of a particular cancer is relatively low in the general population. The majority of available tumor marker tests are not useful in diagnosing cancer in symptomatic patients because elevated levels of markers are also seen in a variety of benign diseases. The main clinical value of tumor markers is in tumor staging, monitoring therapeutic responses, predicting patient outcomes and detecting recurrence of cancer.

Magnetic resonance spectroscopy (MRS) is a technique that has the potential to detect small and early biochemical changes associated with disease processes, and has been proven to be useful in the study of tissue biopsies from cancer patients. It is particularly useful for detecting, in a given biological sample, small, mobile chemical species that are of diagnostic interest. Obtaining tissue. biopsies for such an examination, however, usually involves an invasive procedure. C. L. Lean et al (Magn. Reson Med 20:306-311, 1991; Biochemistry 3:11095-11105, 1992 and Magn Reson Med 30:525-533, 1992) describe the use of magnetic resonance spectroscopy to examine colon cells and tissue specimens. Bezabeh et al in WO 00/71997 and in WO 02/12879 describe a method of diagnosing colorectal adenomas and cancer using MRS on stool samples. WO 00/71997 teaches the use of MRS to identify specific chemical species such as fucose that may be indicative of colon cancer and polyps. WO 02/12879 teaches the use of MRS and a classification-based strategy to differentiate between diseased and non-diseased patients based on their stool. Although useful, MRS requires expensive equipment.

An objective of the present invention is to provide relatively low cost alternative method for detecting small and early biochemical changes associated with colorectal disease processes, and in particular adenomas and cancer.

Another objective of the invention is to provide a sensitive, specific, safe method of detecting presence of colorectal adenomas or cancer in a patient.

Accordingly, the present invention relates generally to a method of detecting colorectal adenomas and cancer in a patient comprising the steps of subjecting a stool sample from the patient to infrared spectroscopy; and comparing the resulting spectrum with infrared spectra of stool from non-cancerous subjects, observed differences in spectra being indicative of cancer or clinically significant adenomas.

The infrared (IR) region of light is in between the visible and microwave portions of the electromagnetic spectrum. The IR spectral region ranges from 780 to 25,000 nm (12800 cm⁻¹ to 400 cm⁻¹) and is commonly subdivided into further regions including the near-IR (4000-12800 cm⁻¹) and mid-IR (400-4000 cm⁻¹). IR spectroscopy (IRS) measures the absorption of infrared radiation by chemical bonds. Therefore, IR spectra contain the basic vibrational fingerprints of all molecules examined in a particular sample and this information can provide insight on the nature of the chemical bonds, the structure and the microenvironment of the sample being studied.

Fragments of molecules, known as functional groups, tend to absorb IR radiation in the same frequency, regardless of the structure of the rest of the molecule containing the functional group. For example, absorptions between 1620-1680 cm⁻¹ are usually attributed to the amide I vibration of proteins, while absorptions at 1080 and 1240cm⁻¹ are attributed to the PO₂ -symmetric and asymmetric stretching vibrations of DNA phosphodiester groups.

Infrared spectroscopy can be used to study substances such as carbohydrates, proteins, lipids and DNA in isolation or as part of complex biological samples. Such biological samples include tissues (for example, whole tissues in vivo or ex-vivo, tissue slices, histological sections and cell suspensions) and fluids (for example, urine, blood, amniotic fluid), even if the fluids are first dried onto an IR-compatible substrate.

IRS can be used in various modalities to study biological samples, including transmission, attenuated total reflectance, diffuse reflectance and Raman Spectroscopy. Data processing techniques such as spectral subtraction, spectral derivatives, deconvolution, multivariate analysis (such as linear discriminate analysis and partial least squares regression) and unsupervised methods (such as principal components analysis and various clustering techniques) are then used to analyze the complex IR spectroscopic data.

IRS can be performed with relatively inexpensive equipment. It has been used for clinical chemistry applications with IR-transparent substrates such as barium fluoride, and with substrates that have limited IR-transparency such as glass, demonstrating its utility and its potential as a cost-effective modality for mass-screening.

IRS has been proven to be useful in the study of tissue biopsies from cancer patients including tissue samples from patients with colon cancer. Human colon adenocarcinoma cell lines display infrared spectroscopic features of malignant colon tissues.

These findings have been extended to the in-vivo and ex-vivo analysis of colon polyps by near infrared Raman spectroscopy and multivariate statistical techniques.

IRS analysis has also been used to screen for colon cancer by the fecal occult blood test by optically detecting the presence of blood in smeared stool samples and IRS has been used to assess the location of gastric bleeding based on the spectroscopic analysis of centrifuged stool samples by means of an artificial neural net.

IRS has also been used on stool to assess nutrient uptake by measuring fecal polyethylene glycol, fecal fat levels, etc., all by measuring known chemicals at specific peaks.

In one embodiment of the method, the stool sample is mixed with a buffer to produce a suspension of stool sample, the suspension is centrifuged to yield a supernatant sample, the supernatant sample is subjected to infrared spectroscopy, and the resulting spectrum is compared with infrared spectra of stool from non-cancerous subjects.

Performing spectral analysis on human stool offers a significant advantage over other methods, because the collection of the specimen is non-invasive and presents no risk to the patient.

Stool samples were collected at the University of Texas M.D. Anderson Cancer Center; University of Manitoba, Health Sciences Centre; University of Chicago; and University of Toronto, Mount Sinai Hospital. Subjects were instructed to collect their bowel movements prior to their colonic preparations. The samples were kept frozen in the patients' refrigerators for an average of 24-48 hours prior to their delivery to the hospital in small ice chests (mailers). They were then stored in a −70 degrees Centigrade freezer until being shipped “blinded”, on dry ice, to the National Research Council Institute for Biodiagnostics, Winnipeg, Canada. All samples were shipped in dry ice and kept frozen at −70 degrees Centigrade until the time of the experiment. There was no significant difference in the lengths of time for which the samples were kept frozen. All samples were randomly assigned a code number that was not traceable to the original sample.

Sample Preparation

For IRS experiments, samples were thawed and a portion of the sample was then taken and suspended in saline. The suspension was then gently vortexed, and replicate dry films were prepared by depositing about 5 μl of the suspension on an infrared-transparent (barium fluoride-BaF₂) window and drying it down quickly under mild vacuum as a thin circular film of 2-3 mm diameter. The remaining sample was then centrifuged and replicate films were prepared by drying 15 μl aliquots onto BaF₂ windows. After measurements, the materials in the windows were washed out with 70% alcohol and water and the waste was stored at the biohazard container. During preparation, the operator wore gloves throughout the procedures to avoid any potential contamination.

IRS Experiments

For each sample, single beam IR spectra were ratioed against the spectrum of a blank barium fluoride window and converted to absorbance units. All spectra were acquired using a Bio-Rad FTS-60 IR spectrometer equipped with a nitrogen cooled mercury cadmium telluride detector, set at a nominal resolution of 2 cm⁻¹ and an encoding interval of one wavenumber. For each spectrum, 256 interferograms were co-added and apodized with a triangular smoothing function before Fourier transformation. Each sample was run twice, resulting in two replicate spectra. This made it possible to check for inconsistencies in the IR processing.

Data Processing

A region consisting of 1,608 data points from each spectrum was used for the analysis. This covered most of the mid-IR range, from 900cm⁻¹ to 4000 cm⁻¹. Each spectrum was then normalized by dividing every data point by the total spectral area. Depending on the data set, it may be advantageous to perform further processing according to methods known to those skilled in the art in light of the disclosure herein. By taking first derivatives, offsets between the spectra were eliminated. The first derivative used simply replaced each data point by the difference between it and the adjacent data point. Performing this operation a second time yielded a second derivative, which eliminated any differences in baseline slopes between spectra. After either derivative is taken, or even if no derivative is used, one may rank order the spectral intensities, replacing the smallest intensity by 1, second smallest by 2, and so on up to the largest intensity, replaced by N, where N is the number of intensity values. This can help in making robust any methods to discriminate between the classes of data, by keeping all the data within the same bounds. A spectrum that originally contained a very large peak (outlier) did not appear as great an outlier to a classifier after rank ordering.

The statistical classification strategy used has been developed specifically to deal with the discrimination of spectra of biomedical origin. The strategy comprises three stages. The first stage is a preprocessing step, found to be preferred for reliable classification. It consists of selecting from the spectra a few maximally discriminatory subregions, using an optimal region selection (ORS) algorithm, based on a genetic algorithm (GA)-driven optimization method (A. E. Nikulin et al, NMR in Biomedicine 11, 209-217 (1998), Near-optimal Region Selection for Feature Space Reduction: Novel Preprocessing Methods for Classifying MR spectra; T. Bezabeh et al, The Use of ¹H Magnetic Resonance Spectroscopy in Inflammatory Bowel Disease: Distinguishing Ulcerative Colotis from Crohn's Disease, Am. J. Gastroenterol 2001, 96: 442-448; R. L. Somorjai et al, Distinguishing Normal from Rejecting Renal Allographs: Application of a Three-Stage Classification Strategy to MR and IR Spectra of Urine, Vibrational Spectroscopy 28 (1) 97-102 (2002), C. L. Lean et al, Accurate Diagnosis and Prognosis of Human Cancers by Proton MRS and a Three-stage Classification Strategy, Annual Reports on NMR Spectroscopy 2002, 48: 71-111) and R. L. Somorjai et al, A Data-Driven, Flexible Machine Learning Strategy for the Classification of Biomedical Data in “Artificial Intelligence Methods and Tools for Systems Biology, Azuaje F. Dubitzby W (eds), Boston: Kluwer Academic Publishers (in press). For reliability of classification, the number of these subregions are preferably an order of magnitude smaller than the number of samples to be classified.

The ORS algorithm was run several times using different starting points on each of several different random splits of the data. For each split, roughly ⅔rds of the samples (two replicate spectra each) were selected for the training set (used to construct the classifier), and the remainder were used as a test set (to estimate the classifier's prediction accuracy on new samples). This method of several random splits is preferable to using just one training and test set, as inevitably some training sets will be more representative than others of the entire possible data space. Classifiers trained using these data sets will generally show higher accuracies on the test samples.

Generally ⅔rds of the samples in the smallest class are selected for training, and then an equal number of samples (may be a smaller percentage) in the larger class are selected. This eliminates any significant imbalances in the number of samples for each class; a large class cannot overwhelm a smaller one (and make it more difficult to classify). Nevertheless, if one class still proves much more difficult to classify than the other, that class can be given more weight, making it more important in scoring the subregions.

Due to the non-exhaustive nature of the ORS algorithm, it is entirely possible that certain subregions from one data split, when combined with subregions from another data split, will yield higher classification accuracies than when used alone. Investigators may collect a large number of promising subregions, and then exhaustively search through all possible subsets for a small number of subregions that still yields good classification accuracy. As already stated, the number of subregions should be kept small for reliability of classification.

Once a set of optimal subregions has been found, the second stage involves computing the ultimate classifier based on those regions. To avoid the overly optimistic classification results that a straight resubstitution approach would give, the inventors have developed a cross-validation method, using a bootstrap methodology. The bootstrap method repeatedly partitions (with replacement) the data into many approximately equal sized random training and test subsets. For each of the random training subsets an optimal classifier is found, and its accuracy is validated on the random test subset. The process is repeated a number of times, usually 10,000. The ultimate classifier is a weighted average of the classifier coefficients of the 10,000 individual component classifiers. This approach effectively uses all n samples.

A standard multivariate statistical method, Linear Discriminant Analysis (LDA) is the preferred choice for all classifiers at all stages, because of its speed and robustness. The concept of crispness of a classifier is also used because the inventors' classifiers produce class probabilities. As used herein, a 2-class classification of a sample is considered crisp if the class assignment probability for that sample is >75%. This crispness is used in the weighting of the classifier coefficients at the bootstrap stage—the weight includes the percentage of samples crisply classified, and Cohen's Kappa (k(0.5,0)), the latter being a measure that indicates the goodness of classification above chance. Similar measures are also used when scoring classifiers at the ORS stage. Generally, subregions producing classifiers with high crispness and Cohen's Kappa values on the test sets are chosen as the optimal ones. Optionally, a penalty function can be used to help minimize the difference in accuracies between the normal and cancer classes.

For difficult classification problems, a third stage consists of combining the outcomes of several classifiers via aggregation methods into an overall classifier that is more reliable and accurate than the individual classifiers.

The particular classifier aggregation used by the inventors is one of the variants of Wolpert's Stacked Generalizer (WSG) (D. H. Wolpert, Stacked Generalization. Neural Networks 5, 241-259 (1992)). The version of WSG used takes the output class probabilities obtained by the individual classifiers as input features to the ultimate classifier. For 2-class problems, the number of features is 1 per classifier (with K independent classifiers this gives K probabilities as input features). The overall classification quality is generally higher. The crispness of the classifier is greater. This is important in a clinical environment because fewer patients will have to be re-examined.

10 regions obtained from an earlier classifier development were used to produce the results reported by using 1st derivatives, 1st derivatives rank ordered, 2nd derivatives and 2nd derivatives rank ordered (4 different classifiers). The probabilities produced by these 4 classifiers were then combined by stacked generalization. 10 random splits of the data were made.

The earlier classifies development involved magnetic resonance (MR) spectra.

The MR spectra from which the classifiers were developed consisted of 324 Normals and 73 Cancers.

| | All samples | Crisp | % Crisp |
|---|---|---|---|
| SE | 83.6% | 86.2% | 63.0% |
| SP | 79.9% | 91.3% | 64.8% |
| Acc | 80.6% | 87.1% | 64.5% |

The IR spectra from which the current classifiers were developed consisted of 393 Normals and 70 Cancers.

| | All samples | Crisp | % Crisp |
|---|---|---|---|
| SE | 84.3% | 91.9% | 52.9% |
| SP | 83.2% | 91.4% | 64.9% |
| Acc | 83.4% | 91.4% | 63.1% |

The number of samples common to the two spectroscopic modalities is 301 Normals and 55 Cancers.

Applying the MR classifier to the common samples gives

| | All samples | Crisp | % Crisp |
|---|---|---|---|
| SE | 80.0% | 88.2% | 61.8% |
| SP | 79.4% | 86.0% | 64.1% |
| Acc | 79.5% | 86.3% | 63.8% |

Applying the IR classifier to the common samples gives

| | All samples | Crisp | % Crisp |
|---|---|---|---|
| SE | 83.6% | 93.1% | 52.7% |
| SP | 83.7% | 90.4% | 65.8% |
| Acc | 83.7% | 90.7% | 63.8% |

Combining the MR and IR classifier probabilities via Wolpert gives (0.01%)

| | All samples | Crisp | % Crisp |
|---|---|---|---|
| SE | 89.1% | 89.6% | 87.3% |
| SP | 87.7% | 92.8% | 82.7% |
| Acc | 87.9% | 92.3% | 83.4% |

Combining the MR and IR classifier probabilities via Wolpert gives (0.01 %)

| | All samples | Crisp | % Crisp |
|---|---|---|---|
| SE | 89.1% | 89.6% | 87.3% |
| SP | 87.7% | 92.8% | 82.7% |
| Acc | 87.9% | 92.3% | 83.4% |

In the above “SE” means sensitivity (an operating characteristic of a diagnostic test that measures the ability of the test to detect a disease or condition when it is truly present). Sensitivity is the proportion of all diseased patients for whom there is a positive test, determined as the number of true positives divided by the sum of true positives plus false negatives. “SP” stands for specificity (a statistical measure of the accuracy of a screening test, i.e. how likely a test is to label as a negative those who do not have a disease or condition, and “Acc” means accuracy. The term “Normals” includes some subjects with colonic conditions/abnormalities that are non-neoplastic. Examples include diverticulosis, hyperplastic polyps and internal hemorrhoids. Specimens with inflammatory bowel disease were not included in the analysis.

The foregoing provides substantive proof that IRS of stool samples can be used effectively to detect the presence of clinically significant adenomas or colorectal cancer.

While the invention, as described above subjects a suspension of a stool sample to IRS, it is also possible to subject a stool sample itself to IRS or to mix a sample with a buffer to form a suspension, centrifuge the suspension to yield a supernatant sample, and subject the sample to IRS.

The inventors have also determined that the use of the method of the present invention in combination with the method described in applicants' earlier applications, WO 02/12879 (supra) or WO 04/027419 (Bezabeh) results in a more conclusive test for the presence of colorectal cancer and/or clinically significantly adenomas. The earlier methods involve the use of magnetic resonance spectroscopy (MRS). The simultaneous performance of the two tests (MRS and IRS) aliquots of a stool sample would provide a better indication of the presence of cancer or adenomas.

1. A method of detecting colorectal adenomas and cancer in a patient comprising the steps of subjecting a stool sample from the patient to infrared spectroscopy; and comparing the resulting spectrum with infrared spectra of stool from non-cancerous subjects, observed differences in spectra being indicative of cancer or clinically significant adenomas.

2. The method of claim 1, including the steps of preparing a liquid suspension of the stool samples, and subjecting the suspension to infrared spectroscopy.

3. The method of claim 2, wherein the liquid suspension is a saline suspension of the stool sample.

4. The method of claim 1, wherein the stool sample is mixed with a buffer to produce a suspension; the suspension is centrifuged to yield a supernatant; and the supernatant is subjected to infrared spectroscopy.

5. The method of claim 1, including the steps of selecting subregions from the spectra of stool that are maximally discriminatory between non-cancerous and cancerous subjects; repeatedly partitioning data thus obtained into approximately equal sized random training and test subsets; finding an optimal classifier for each random training subset; validating the accuracy of the optimal classifier on the random test subset; and determining the ultimate classifier as the weighted average of the classifier coefficients of a large number of individual component classifiers.
